(19) United States
(12) Patent Application Publication
(10) Pub. No.: US 2026/0263357 A1
(43) Pub. Date: Sep. 10, 2026

(54) PHASE SHIFTING INTRAVITREAL TAMPONADE

(71) Applicant: Avant Sciences, Inc., New York, NY (US)

(72) Inventor: Avnish Arvind Deobhakta, New York, NY (US)

(21) Appl. No.: 19/473,292

(22) PCT Filed: Apr. 9, 2024
(86) PCT No.: PCT/US2024/023759

§ 371 Date: Oct. 7, 2025

Related U.S. Application Data
(60) Provisional application No. 63/495,262, filed on Apr. 10, 2023.

(51) Int. Cl. A61K 9/00; A61K 31/08; A61P 27/02

(52) U.S. Cl. CPC A61K 9/0051; A61K 9/0024; A61K 31/08; A61P 27/02

(57) ABSTRACT

A vitreous humor tamponade for the posterior cavity in the eye, including at least one structurally asymmetric fluorinated ether which is a liquid during room temperature or operative conditions and which can, after placement into the eye, change into gas as a result of its native vapor pressure or via external non-invasive methods. Such a compound also has a specific gravity between 1.1 and 1.6 (the latter being an upper bound to prevent retinal toxicity), is immiscible in water, is non-combustible, has high ultrasonic absorption capabilities, and is clear and colorless, in addition to the requirement described above.

### INCORPORATION BY REFERENCE TO ANY PRIORITY APPLICATIONS

This application claims the benefit of U.S. Provisional Ser. No. 63/495,262, filed Apr. 10, 2023, which is hereby incorporated by reference in its entirety.

### BACKGROUND

### Field of the Disclosure

Aspects of the present disclosure generally relate to retinal tamponades and methods of use thereof. More specifically, some aspects of the present disclosure relate to phase shifting intravitreal tamponades, including liquid intraocular tamponade agents for treating inferior retinal detachments, and methods of use thereof.

### Description of the Related Art

Retinal detachments or tears often require the need for some type of intraocular tamponade agent post intervention in order for successful reattachment. In order to fix a retinal detachment caused by a retinal tear, the retina is flattened around the tear, a scarring procedure is placed to “seal” the retinal tear, and an intraocular tamponade compound usually is necessary to keep the retina flat and in place against the wall of the eye for some period of time while healing and scarring occurs. Currently, the type of tamponade agent used is specific to both the intervention done (surgery done in the operative theater or non-surgical procedure done in the clinic) and where the retinal tear and its subsequent detachment is located relative to the eye.

For retinal detachments due to superior retinal tears, a non-surgical intervention is often attempted. Typically, a gaseous perfluorinated agent (frequently either perfluoropropane or sulfur hexafluoride) is placed in the vitreous cavity and is selected for its high surface tension and low density relative to intraocular fluid. This agent is able to effectively “float” above the intraocular fluid and provide a sufficient amount of upward force against the detached retina and a high enough surface tension to close the retinal tear, thus allowing the retina to flatten and permitting a permanent scarring treatment to be placed around the retinal tear, usually via thermal laser burns. The gaseous perfluorinated agent can then either be vented out using a bedside needling procedure or is allowed reabsorb on its own over time via the blood stream and then exit the body. As a result, surgery in the operative theater can often be avoided, reducing both the morbidity burden on the patient and the financial costs inherent to operating room procedures to society at large.

However, for retinal detachments due to inferior retinal tears, an intraocular tamponade agent would require a density higher than that of native intraocular fluid/vitreous (in order for it to “sink”). Previous attempts to solve this problem have used perfluorinated or partially fluorinated liquids, which offer downward tamponade due to having a specific gravity larger than 1, and usually closer to 2, but these liquids require surgical intervention in the operating theater to safely remove once placed. In addition, such liquids do not provide sufficient upward tamponade unless the entire vitreous cavity is filled with the compound, which further necessitates surgical intervention to place (and remove) safely. Indeed, during intraoperative surgery to fix retinal detachments, an intraocular perfluorinated liquid tamponade agent (n-perfluro-octane) is often used to stabilize the retina. This compound, however, must be removed after surgery due to both toxic effects of long-term use inherent to the compound and the lack of a practical way to remove the fluid without returning to the operating room. Due to the lack of a possible non-operative room procedural fix, and the inherently difficult nature of providing long-term intraocular tamponade to this area for healing, inferior retinal detachments are a large source of morbidity for patients as re-detachment after surgery for inferior retinal detachments is far more frequent compared to other types of retina detachment.

### SUMMARY

Some aspects of the present disclosure relate to retinal tamponade agent. In some embodiments, the retinal tamponade agent comprises a structurally asymmetric partially fluorinated ether having Structure 1:

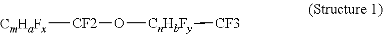

wherein m is 0, 1, or 2, or 3; a is 0, 1, or 2; x is 0, 1, 2, 3, 4, 5, or 6; n is 1 or 2, b is 1 or 2; and y is 0, 1, 2, 3, or 4.

In some embodiments, m is 1, a is 1, x is 2, n is 1, b is 2, and y is 0. In some embodiments, the structurally asymmetric partially fluorinated ether comprises 2,2,2-Trifluoroethyl 1,1,2,2-tetrafluoroethyl ether.

In some embodiments, m is 0, a is 1, x is 0, n is 2, b is 1, and y is 3. In some embodiments, the structurally asymmetric partially fluorinated ether comprises 1,1,2,3,3,3-Hexafluoropropyl difluoromethyl ether.

In some embodiments, m is 2, a is 0, x is 5, n is 1, b is 1, and y is 1. In some embodiments, the structurally asymmetric partially fluorinated ether comprises Heptafluoropropyl 1,2,2,2-tetrafluoroethyl ether.

In some embodiments, m is 0, a is 1, x is 0, n is 2, b is 2, and y is 2. In some embodiments, the structurally asymmetric partially fluorinated ether comprises Difluoromethyl 2,2,3,3,3-pentafluoropropyl ether.

In some embodiments, the structurally asymmetric partially fluorinated ether is chiral. In some embodiments, the structurally asymmetric partially fluorinated ether is asymmetric around the rotational axis. In some embodiments, the structurally asymmetric partially fluorinated ether changes from liquid to gaseous form at native vapor pressure. In some embodiments, the structurally asymmetric partially fluorinated ether has a boiling point of between about 36° C. and 58° C., and a specific gravity of less than about 1.6 g/cm³. In some embodiments, the structurally asymmetric partially fluorinated ether has an amplitude attenuation coefficient (db/cm-Hz) that is greater than the amplitude attenuation coefficient (db/cm-Hz) of water. In some embodiments, the agent comprises an amplitude attenuation coefficient (db/cm-Hz) that is greater than 0.002 db/cm-MHz. In some embodiments, the structurally asymmetric partially fluorinated ether comprises an amplitude attenuation coefficient (db/cm-Hz) that is between about 0.003-4 db/cm-MHz. In some embodiments, the structurally asymmetric partially fluorinated ether is immiscible in water. In some embodiments, the structurally asymmetric partially fluorinated ether is non-combustible between about 36° C. and 58° C. In some embodiments, the structurally asymmetric partially fluorinated ether is optically clear in liquid form. In some embodiments, the structurally asymmetric partially fluorinated ether is optically clear in gaseous form. In some embodiments, the structurally asymmetric partially fluorinated ether changes from liquid to gaseous form at native vapor pressure. In some embodiments, the structurally asymmetric partially fluorinated ether changes from liquid to gaseous form in about 1-3 days post administration into the vitreous chamber under biological conditions. In some embodiments, the structurally asymmetric partially fluorinated ether changes from liquid to gaseous form in about 1 week post administration into the vitreous chamber under biological conditions. In some embodiments, the structurally asymmetric partially fluorinated ether does not change from a liquid to a gaseous form unless external energy is applied. In some embodiments, the structurally asymmetric partially fluorinated ether is carried out of the body over within about 8 days of the change from liquid to gaseous form. In some embodiments, the structurally asymmetric partially fluorinated ether is carried out of the body over within about 1 month of the change from liquid to gaseous form. In some embodiments, the structurally asymmetric partially fluorinated ether is carried out of the body over within about 2 months of the change from liquid to gaseous form. In some embodiments, the structurally asymmetric partially fluorinated ether is non-toxic to intraocular structures when left in place for greater than 1 month. In some embodiments, the structurally asymmetric partially fluorinated ether is non-toxic to intraocular structures when left in place for greater than 6 months.

Some aspects of the present disclosure relate to a method of treating retinal detachment. In some embodiments, the method comprises administering into a vitreous chamber of an eye an effective amount of the retinal tamponade agent comprising Structure 1. In some embodiments, the retinal detachment is due to an inferior retinal tear. In some embodiments, the retinal detachment is due to a superior retinal tear.

### BRIEF DESCRIPTION OF THE DRAWINGS

FIG. 1A shows examples of retinal fundus images of rabbit eyes before and up to about 2 months after balanced salt solution was injected into the vitreous cavity.

FIG. 1B shows examples of retinal fundus images of rabbit eyes before and up to about 2 months after tamponade was injected into the vitreous cavity.

FIG. 2 is a plot of measured intraocular pressure of the rabbit eyes before and up to about two months after balanced salt solution and tamponade were injected into the vitreous cavity.

FIG. 3 shows scotopic a-wave measurements of rabbit eyes before and about two months after injecting a balanced salt solution and tamponade into the vitreous cavity.

FIG. 4 shows scotopic b-wave measurements of rabbit eyes before and about two months after injecting a balanced salt solution and tamponade into the vitreous cavity.

FIG. 5 shows photopic b-wave ERGs of rabbit eyes before and about two months after injecting a balanced salt solution and tamponade into the vitreous cavity.

FIG. 6A shows examples of retinal fundus images of rabbit eyes before and up to about 1 months after balanced salt solution and tamponade were injected into the vitreous cavity.

FIG. 6B shows examples of fundus images from a single rabbit in which 8 microliters of 1,1,2,3,3,3-Hexafluoropropyl difluoromethyl ether (EOD-2) were injected into the vitreous cavity.

FIG. 7 is a graph showing intraocular pressures of eyes injected with 1,1,2,3,3,3-Hexafluoropropyl difluoromethyl ether (EOD-2) or a balanced salt solution (BSS).

### DETAILED DESCRIPTION

Some embodiments herein are directed to a liquid intraocular tamponade agent that provides downward tamponade to the inferior retina that has the ability to convert to gaseous phase safely within the eye. Some embodiments herein are directed to methods of treating retinal detachment, including superior and inferior retinal detachments without a visit to the operating room, as it could be removed via either venting or via bloodstream absorption after reattachment and scarring treatment.

Disclosed herein includes some embodiments of a liquid intraocular tamponade agent that could provide downward tamponade to the inferior retina and has the ability to convert to gaseous phase safely within the eye. Some embodiments are directed to methods of treating retinal detachment, including superior and inferior retinal detachments, using the liquid intraocular tamponade agent disclose herein. In some embodiments the retinal detachment may treated without a visit to the operating room, as the tamponade agent disclosed herein could be removed via either venting or via bloodstream absorption after reattachment and scarring treatment. Such an agent, while in gaseous form, would also have the added benefit of providing superior retinal tamponade. In addition, if the specific gravity is within a specific range, it might be useful as an intraoperative tamponade that could function like the liquids in use today (n-perfluoro-octane) but could be left in the eye and converted to gas either gradually or when needed for removal without a further trip to the operating room. In some embodiments, the tamponade agent may be clear, immiscible in water, biologically non-toxic, and/or without a flash point.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

As used herein, the term “retina” refers to the light-sensitive layer of tissue at the back of the eyeball.

As used herein, the term “retinal detachment” refers to a condition in which the retina pulls away from its normal position. Retinal detachment separates the retinal cells from the layer of blood vessels that provides oxygen and nourishment to the eye. The longer retinal detachment goes untreated, the greater the risk of permanent vision loss in the affected eye.

As used herein, the term “tamponade” refers to the provision of temporary or permanent surface tension across retinal breaks.

As used herein, the term “flash point” refers to the temperature at which a particular organic compound gives off sufficient vapor to ignite in air.

### Liquid Tamponade Agents

Perfluorinated or partially fluorinated alkanes/ethers with the capacity to phase shift at under biological conditions are often flammable and thus dangerous to use in a medical context. Generally speaking, as a perfluorinated chemical becomes less fluorinated, the boiling point increases until it hits a critical mass of hydrogen substitutions, at which point it begins to decrease again. Hence, a potential hydrogen “substituted in for fluorine” analogue chemical to the stable gaseous agent perfluoropropane (C3F8)—which itself has a low boiling point—may be useful up to a certain amount of substitutions if shown to be medically safe. Unfortunately, as shown below in Table 1, the less fluorinated analogues that have a boiling point near biologically active ranges have an increasing propensity to explode at handling, room, body, and reasonable environmental temperature.

TABLE 1
Selected Fluorinated Alkanes

| | Chemical | Boiling Point | |
|---|---|---|---|
| Chemical Name | Formula | (° C.) | Flammable? |
| Perfluoropropane | C3F8 | −39 | No |
| 1,1,1,2,2,3,3-Heptafluoropropane | C3F7H | −16 | No |
| 1, 1,2,2,3,3-Hexafluoropropane | C3F6H2 | 5 | No |
| 1,1,2,2,3-Pentafluropropane | C3F5H3 | 26 | YES |
| 1,1,1,3-Tetrafluoropropane | C3F4H4 | 30 | YES |
| 1,1,3-Trifluoropropane | C3F3H5 | 45 | YES |

Additionally, while partially fluorinated ethers are not in routine use for retinal surgery, the potential efficacy for retinal tamponade of some of them have also been described. Yet, while these chemicals would seem ideal for this particular use, most have similar flammability issues with analogues that approach biologically active ranges, as shown in Table 2.

TABLE 2
Selected Fluorinated Ethers

| | | Boiling | |
|---|---|---|---|
| | Chemical | Point | |
| Chemical Name | Formula | (° C.) | Flammable? |
| Ethyl pentafluoroethyl ether | C4H5F5O | 30 | YES |
| Bis(fluoromethyl) ether | C2H4F2O | 34 | YES |
| Heptafluoropropyl methyl ether | C4H3F7O | 34 | No |
| tetrafluoroethyl methyl ether | C3H4F4O | 37 | YES |
| 1,1-Difluoroethyl 2,2,2- | C4H5F5O | 39 | YES |
| trifluoroethyl ether* | | | |
| 1,2,2,2-Tetrafluoroethyl methyl | C3H4F4O | 39 | YES |
| ether* | | | |
| Heptafluoropropyl 1,2,2,2- | CSHF11O | 40 | No |
| tetrafluoroethyl ether* | | | |
| Difluoromethyl 2,2,3,3,3- | C4H3F7O | 46 | No |
| pentafluoropropyl ether* | | | |
| 1,1,2,3,3,3-Hexafluoropropyl | C4H2F8O | 47 | No |
| difluoromethyl ether* | | | |
| 2,2,2-Trifluoroethyl 1,1,2,2- | C4H3F7O | 56 | No |
| tetrafluoroethyl ether* | | | |

*Denotes BP in range for intraocular use

However, a small number of fluorinated alkanes/ethers were found that may be both safe and with a boiling point at around biologically active ranges (as an example, Heptafluoropropyl 1,2,2,2-tetrafluoroethyl ether in Table 2). It is likely that these compounds match the criterion needed because a perfluoropropane-like (C3F8) moiety exists on one part of the ether substructure (see circled portion of Structure A, below) which likely lends stability while still having enough carbons and hydrogen “substitutions” on the second part of the ether substructure to have an in-range boiling point. However, while one of the other non-flammable ethers in Table 2 (Heptafluoropropyl methyl ether, Structure B) also possesses a similar perfluoropropane-like moiety, it does not possess the correct combination of carbons and/or hydrogen/fluorine “substitutions” to have as useful of a boiling point for this use. In fact, one R group of the heptafluoropropyl methyl ether has no fluorine on it at all (see circled portion of Structure B, below). In some embodiments, ethers having multiple fluorine substitutions on both alkyl chains and having boiling points within biologically active range may be useful as tamponades that have desirable qualities discussed herein.

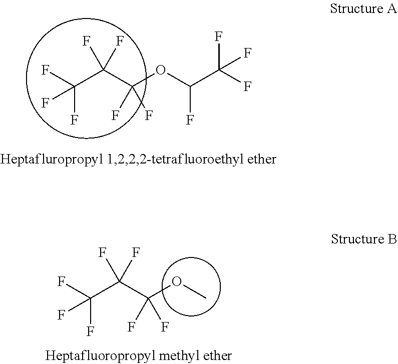

Some aspects of the present disclosure are directed to structurally asymmetric partially fluorinated ethers. In some embodiments, the compounds have the following structure:

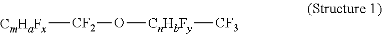

wherein m is 0, 1, 2, or 3, a is 0, 1, or 2, x is 0, 1, 2, 3, 4, 5, or 6, n, b is 1 or 2, y is 0, 1, 2, 3, or 4. In some embodiments, the tamponade agent has a boiling point around native human intraocular temperature. In some embodiments, the tamponade agent has a specific gravity of approximately 1.1 to approximately 1.6. In some embodiments, the tamponade agent is immiscible in water. In some embodiments, the tamponade agent is optically clear in liquid form. In some embodiments, the tamponade agent is optically clear in gaseous form. In some embodiments, the tamponade agent has a high surface tension upon gaseous phase shift. In some embodiments, tamponade agent has a high ultrasonic absorption characteristic. In some embodiments, the tamponade agent is non-toxic to intraocular structures. In some embodiments the tamponade agent does not have a flash point. In some embodiments, the tamponade agent is not flammable under the conditions it is being handled, stored, and used in.

In some embodiments, the tamponade agent has a boiling point around native human intraocular temperature. In some embodiments, the tamponade agent has a boiling point of about 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C., or a boiling point in a range defined by any two of the preceding values. For example, in some embodiments, the tamponade agent comprises a boiling point of between about 35° C.-about 40° C., about 36° C.-about 40° C., about 36° C.-about 39° C., about 36° C.-about 38° C.

In some embodiments, the tamponade agent comprises a specific gravity of 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, or 2 g/cm³, or a specific gravity that is in a range defined by any two of the preceding values. For example, in some embodiments, the tamponade agent comprises a specific gravity of between about 0.8-2 g/cm³, 0.8-1.8 g/cm³, 0.8-1.6 g/cm³, 0.8-1.4 g/cm³, 0.8-1.2 g/cm³, 0.8-1 g/cm³, 1-2 g/cm³, 1-1.8 g/cm³, 1-1.6 g/cm³, 1-1.4 g/cm³, 1-1.2 g/cm³, 1-1.1 g/cm³, 1.1-2 g/cm³, 1.1-1.8 g/cm³, 1.1-1.6 g/cm³, 1.1-1.4 g/cm³, 1.1-1.2 g/cm³, 1.2-2 g/cm³, 1.2-1.8 g/cm³, 1.2-1.6 g/cm³, 1.2-1.4 g/cm³, 1.4-2 g/cm³, 1.4-1.8 g/cm³, 1.4-1.6 g/cm³, 1.6-2 g/cm³, 1.6-1.8 g/cm³, or 1.8-2 g/cm³.

In some embodiments the tamponade agent does not have a flash point. In some embodiments, the tamponade agent is not flammable under the conditions it is being handled, stored, and used in. In some embodiments, the tamponade agent comprises a flash point that is greater than about 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. In some embodiments, the tamponade agent comprises a flash point that is above about 60° C.

In some embodiments the tamponade agent changes from liquid to gaseous form in about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days post administration into the vitreous chamber under biological conditions, or in a number of days that is in a range defined by any two of the preceding values. For example, in some embodiments, the tamponade agent changes from liquid to gaseous form in between about 1-31, 1-30, 1-29, 1-28, 1-21, 1-14, 1-10, 1-7, 1-5, 1-3, 3-31, 3-30, 3-29, 3-28, 3-21, 3-14, 3-10, 3-7, 3-5. 5-31. 5-30, 5-29, 5-28, 5-21, 5-14, 5-10, 5-7, 7-31, 7-30, 7-29, 7-28, 7-21, 7-14, 7-10, 10-31, 10-30, 10-29, 10-28, 10-21, 10-14, 14-31, 14-30, 14-29, 14-28, 14-21, 21-31, 21-30, 21-29, 21-28, or 28-31 days. In some embodiments, the tamponade agent changes from liquid to gaseous form in greater than 31 days. In some embodiments, the tamponade agent does not change from a liquid to a gaseous form unless external energy is applied.

In some embodiments, the tamponade agent is carried out of the body over within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days of the change from liquid to gaseous form, or in a number of days that is in a range defined by any two of the preceding values. For example, in some embodiments, the tamponade agent is carried out of the body over within about 1-31, 1-30, 1-29, 1-28, 1-21, 1-14, 1-10, 1-7, 1-5, 1-3, 3-31, 3-30, 3-29, 3-28, 3-21, 3-14, 3-10, 3-7, 3-5. 5-31. 5-30, 5-29, 5-28, 5-21, 5-14, 5-10, 5-7, 7-31, 7-30, 7-29, 7-28, 7-21, 7-14, 7-10, 10-31, 10-30, 10-29, 10-28, 10-21, 10-14, 14-31, 14-30, 14-29, 14-28, 14-21, 21-31, 21-30, 21-29, 21-28, or 28-31, days of the change from liquid to gaseous form. In some embodiments, the tamponade agent is carried out of the body in greater than 31 days of the change from liquid to gaseous form. In some embodiments, the tamponade agent is not carried out of the body. In some embodiments, the tamponade agent is vented or otherwise removed.

In some embodiments, the liquid tamponade agent is non-toxic to intraocular structures. In some embodiments, the liquid tamponade agent is non-toxic to intraocular structures when left in place for greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, days, or for a number of days that is in a range defined by any two of the preceding values. For example, in some embodiments, the tamponade agent is non-toxic to intraocular structures when left in place for greater than between about 1-31, 1-30, 1-29, 1-28, 1-21, 1-14, 1-10, 1-7, 1-5, 1-3, 3-31, 3-30, 3-29, 3-28, 3-21, 3-14, 3-10, 3-7, 3-5. 5-31. 5-30, 5-29, 5-28, 5-21, 5-14, 5-10, 5-7, 7-31, 7-30, 7-29, 7-28, 7-21, 7-14, 7-10, 10-31, 10-30, 10-29, 10-28, 10-21, 10-14, 14-31, 14-30, 14-29, 14-28, 14-21, 21-31, 21-30, 21-29, 21-28, or 28-31, days. In some embodiments, the tamponade agent is non-toxic to intraocular structures when left in place for greater than about 1, 2, 3, 4, 5, 6, 7, 8, 9, 10, 11, or 12 months, or for a number of months in a range defined by any two of the preceding values. For example, in some embodiments, the tamponade agent is non-toxic to intraocular structures when left in place for greater than between about 1-12, 1-10, 1-8, 1-6, 1-4, 1-2, 2-12, 2-10, 2-8, 2-6, 2-4, 4-12, 4-10, 4-8, 4-6, 6-12, 6-10, 6-8, 8-12, 8-10, or 10-12, months. In some embodiments, the tamponade agent is non-toxic to intraocular structures when left in place for greater than about 1 year.

In some embodiments, the tamponade agent does not increase intraocular pressure over baseline intraocular pressure. In some embodiments, the tamponade agent does not increase intraocular pressure to over about 30 mmHg. In some embodiments, the tamponade agent does not increase intraocular pressure to over about 10, 11, 12, 13, 14, 15, 16, 17, 18, 19, 20, 21, 22, 23, 24, 25, 26, 27, 28, 29, or 30 mmHg, or increase intraocular pressure over a range defined by any two of the preceding values. For example, in some embodiments, the tamponade agent does not increase intraocular pressure to over between about 11-30, 11-26, 11-21, 11-15, 15-30, 15-26, 15-21, 21-30, 21-26, or 26-30 mmHg.

Unlike prior referenced tamponade agents, such tamponade agents with the above qualities retain the ability to provide intraocular tamponade of the retina with a safe amount of posterior and downward force in liquid form, while simultaneously also having the unique ability to gradually change from liquid to gaseous form, via either native vapor pressure characteristics or external means. In some embodiments, the tamponade agent provides a sufficient amount of downward and posterior force for treating inferior retinal detachments should it be needed. While maintained in gaseous form, the tamponade agent can be either permitted to reabsorb into the bloodstream and exit the body on its own or be vented out directly via a suitable method, for example, but not limited to, needling or another bedside or intra-clinic method.

Examples of the useful compounds include, but are not limited to:

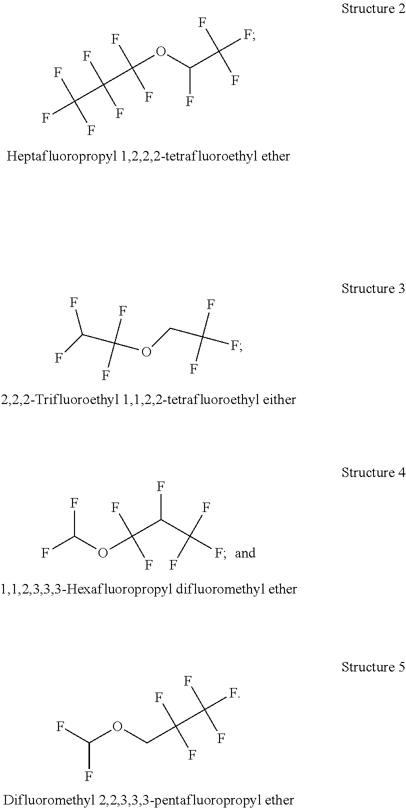 [SMILES: FC(OC(F)(F)C(F)(F)C(F)(F)F)C(F)(F)F ; FC(F)C(F)(F)OCC(F)(F)F ; FC(F)OC(F)(F)C(F)C(F)(F)F ; "and" ; FC(F)OCC(F)(F)C(F)(F)F]

Without being bound to a certain theory, some degree of molecular asymmetry may contribute to the ability for fluorinated ethers to phase shift at around the biologically active temperature (i.e., around body temperature) and without being flammable. In particular, the dimensions of symmetry are granularly defined in molecular chemistry, often involving a combination of geometrical reflection planes, rotational axes, and codified manipulations in 3-dimensional space that discretely categorize the “amount of symmetry” that a given molecule has. To demonstrate this concept, one can compare one of the candidate chemicals from Table 2—1,1,2,3,3,3-Hexafluoropropyl difluoromethyl ether, Structure 4, shown below, with the previously reported fluorinated ether retinal tamponade Decafluoro-di-n-pentyl ether, Structure C (Santos et al., Retina, 33 (1), p 120-127 (1991)), shown below.

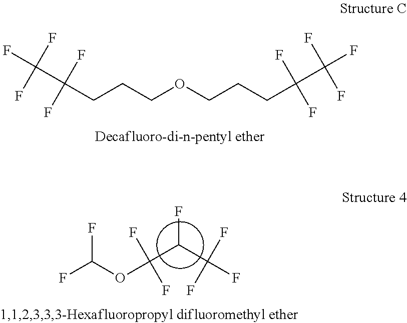 [SMILES: FC(F)(F)C(F)(F)CCCOCCCC(F)(F)C(F)(F)F ; FC(F)OC(F)(F)C(F)C(F)(F)F]

Decafluoro-di-n-pentyl ether, Structure C, is a partially fluorinated ether that has been postulated as a potential retinal tamponade; structurally, it is symmetric across the rotational C2 axis. That is, it can be flipped horizontally, i.e., rotated “around” the central oxygen species 180 degrees as if there were a vertical axis through it, and still maintain the identical structure. In addition, it is symmetric across a mirrored plane; that is, holding this compound up “to a mirror” returns the same chemical in 3-dimensional space that can then be superimposed upon itself to be identical. Indeed, any structure that resembles Decafluoro-di-n-pentyl ether (that is, any structure that adheres to the chemical genus R′(CH₂)ₙO(CH₂)ₙR′, wherein R′ is a fluorinated monovalent saturated organic group and n is a non-negative integer) would share these characteristics. In contrast, 1,1,2,3,3,3-Hexafluoropropyl difluoromethyl ether, Structure 4, does not have an obvious “rotational axis”, as one cannot find an axis in 3-dimensional space around which, if the compound were simply rotated around that axis in some fashion, the identical compound results. In fact, it is so asymmetric that it has a single chiral center; that is, it has a carbon for which there are four different ligands attached to it, in this case a fluorine atom, a tri-fluoromethyl group, a partially fluorinated methoxy group, and a hydrogen atom (circled chiral center). Thus, even mirror symmetry does not exist for this compound. While the existence of chirality is not a necessary component of asymmetry for the purposes described herein, it serves to demonstrate the ability of asymmetric molecules to successfully phase shift safely under biologically active conditions, whilst retaining all other virtuous characteristics of a retinal tamponade.

As a result, decafluoro-di-n-pentyl ether and its analogues are much more phase stable at any given temperature, as their high symmetry allows them to more likely “pack together” in a solution, and thus remain in that phase unless a disproportionately larger amount of energy is employed to change that chemical status quo. This is in distinction to their less symmetric counterpart fluorinated ethers, such as 1,1,2,3,3,3-Hexafluoropropyl difluoromethyl ether and the others detailed at the bottom of Table 2. For the latter molecules, their relatively asymmetric structures permit them to be “less packed together” chemically, and thus, all else held equal, are more likely to shift from liquid to gas under similar energetic conditions.

The compounds of Structure 1 have an asymmetric distribution of multiple fluorines on either side of the central oxygen species, both in number and location. As can be seen above, one part of the molecule is bounded at one end by a fully fluorinated terminal carbon, and the other part of the molecule has fluorination at the carbon bound to the central oxygen species. In some embodiments, the compounds of Structure 1 may be a chiral compound.

In some embodiments, the compounds of Structure 1 are capable of absorbing ultrasonic wave energy. The ultrasonic absorption of some of the compounds is shown in Table 3 below. The capacity of a given chemical to convert applied ultrasound from vibrational energy into heat is termed its ultrasonic absorbance, which itself varies depending upon the initial temperature and the frequency of the applied ultrasound. Additionally, the native vitreous within biological tissues approximates that of water, which itself has an ultrasonic absorption profile; thus, there is a baseline transmission of acoustic energy through ocular tissues, and this characteristic is routinely leveraged when it comes to various forms of imaging and therapy (Southern, Australas J Ultrasound Med. 2009 February; 12 (1): 32-37). In some embodiments, a compound that can preferentially absorb ultrasound more readily than that of water has the capacity to disproportionately absorb applied ultrasonic energy versus that of vitreous within the vitreous cavity. As such absorption would lead to local increase in temperature, thus facilitate the phase shift to a gaseous state.

In some embodiments, tamponade agent has a high ultrasonic absorption characteristic. In some embodiments, the tamponade agent comprises an amplitude attenuation coefficient that is greater than the amplitude attenuation coefficient of water. In some embodiments, the tamponade agent comprises an amplitude attenuation coefficient that is greater than the amplitude attenuation coefficient of vitreous within the vitreous cavity. In some embodiments, the tamponade agent comprises an amplitude attenuation coefficient (db/cm-Hz) that is about 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.1, 2.2, 2.25, 2.3, 2.4, 2.5, 2.6, 2.7, 2.75, 2.8, 2.9, 3.0, 3.1, 3.2, 3.25, 3.3, 3.4, 3.5, 3.6, 3.7, 3.75, 3.8, 3.9, or 4.0 db/cm-Hz, or an amplitude attenuation coefficient that is in a range defined by any two of the preceding values. For example, in some embodiments, the tamponade agent comprises an amplitude attenuation coefficient that is between about 0.002-4, 0.002-3.75, 0.002-3.5, 0.002-3.25, 0.002-3, 0.002-2.75, 0.002-2.5, 0.002-2.25, 0.002-2, 0.002-1.75, 0.002-1.5, 0.002-1.25, 0.002-1, 0.002-0.75, 0.002-0.5, 0.002-0.025, 0.002-0.01, 0.002-0.003, 0.003-4, 0.003-3.75, 0.003-3.5, 0.003-3.25, 0.003-3, 0.003-2.75, 0.003-2.5, 0.003-2.25, 0.003-2, 0.003-1.75, 0.003-1.5, 0.003-1.25, 0.003-1, 0.003-0.75, 0.003-0.5, 0.003-0.025, 0.003-0.01, 0.01-4, 0.01-3.75, 0.01-3.5, 0.01-3.25, 0.01-3, 0.01-2.75, 0.01-2.5, 0.01-2.25, 0.01-2, 0.01-1.75, 0.01-1.5, 0.01-1.25, 0.01-1, 0.01-0.75, 0.01-0.5, 0.01-0.025, 0.5-4, 0.5-3.75, 0.5-3.5, 0.5-3.25, 0.01-3, 0.5-2.75, 0.5-2.5, 0.5-2.25, 0.5-2, 0.5-1.75, 0.5-1.5, 0.5-1.25, 0.5-1, 1-4, 1-3.5, 1-3, 1-2.5, 1-2, 1-1.5, 1.5-4, 1.5-3.5, 1.5-3, 1.5-2.5, 1.5-2, 2-4, 2-3.5, 2-3, 3-4, 3-3.5, or 3.5-4 db/cm-Hz.

TABLE 3
Experimental Ultrasound Absorbance of Selected Compounds

| | Amplitude |
|---|---|
| | Attenuation |
| | Coefficient |
| Chemical Name | (db/cm-MHz) |
| 2,2,2-Trifluoroethyl 1,1,2,2-tetrafluoroethyl ether | 2.65 |
| 1,1,2,3,3,3-Hexafluoropropyl difluoromethyl ether | 3.35 |
| Heptafluoropropyl 1,2,2,2-tetrafluoroethyl ether | 3.58 |
| Difluoromethyl 2,2,3,3,3-pentafluoropropyl ether | 0.57 |
| Water | 0.002 |

With reference to Table 3, a compound with a larger amplitude attenuation coefficient absorbs ultrasound more than a compound that has a lower amplitude attenuation coefficient. It is clear that each of the compounds of Structure 1 (as shown in Table 3) is orders of magnitude more likely to absorb ultrasonic energy than water/vitreous. Furthermore, the two chiral molecules, i.e., 1,1,2,3,3,3-hexafluoropropyl difluoromethyl ether and heptafluoropropyl 1,2,2,2-tetrafluoroethyl ether, have the highest attenuation coefficient, suggesting that increasing asymmetry in a given fluidic compound lends itself to higher ultrasonic absorption. Indeed, it is notable that water, the least absorptive compound shown, is not only clearly symmetric on both a rotational axis and the mirror plane, but also has some of the strongest hydrogen bond forces in all of chemistry, allowing it to “pack itself together” much more readily than the other compounds on the list.

In some embodiments, the tamponade agent comprises Structure 1:

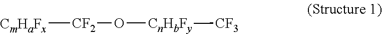

wherein m is 0, 1, 2, or 3, a is 0, 1, or 2, x is 0, 1, 2, 3, 4, 5, or 6, n is 1 or 2, b is 1 or 2, y is 0, 1, 2, 3, or 4. In some embodiments, m is 1, a is 1, x is 2, n is 1, b is 2, and y is 0. In some embodiments, m is 0, a is 1, x is 0, n is 2, b is 1, and y is 3. In some embodiments m is 2, a is 0, x is 5, n is 1, b is 1, and y is 1. In some embodiments, m is 0, a is 1, x is 0, n is 2, b is 2, and y is 2. Table 4, below shows some representative embodiments that illustrate the genus described herein for tamponades.

TABLE 4
Specific Embodiments of Generalized Chemical Structure

| Chemical Name | m | a | x | n | b | y |
|---|---|---|---|---|---|---|
| 2,2,2-Trifluoroethyl 1,1,2,2-tetrafluoroethyl ether | 1 | 1 | 2 | 1 | 2 | 0 |
| 1,1,2,3,3,3-Hexafluoropropyl difluoromethyl ether | 0 | 1 | 0 | 2 | 1 | 3 |
| Heptafluoropropyl 1,2,2,2-tetrafluoroethyl ether | 2 | 0 | 5 | 1 | 1 | 1 |
| Difluoromethyl 2,2,3,3,3-pentafluoropropyl ether | 0 | 1 | 0 | 2 | 2 | 2 |

Some embodiments herein relate to a method of treating retinal detachment. In some embodiments, the method comprises administering into a vitreous chamber of an eye an effective amount of a tamponade agent comprising Structure 1:

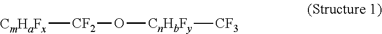

wherein m is 0, 1, 2, or 3, a is 0, 1, or 2, x is 0, 1, 2, 3, 4, 5, or 6, n is 1 or 2, b is 1 or 2, y is 0, 1, 2, 3, or 4. In some embodiments, the retinal detachment is due to an inferior retinal tear. In some embodiments, the retinal detachment is due to a superior retinal tear.

### Numbered Arrangements

Some embodiments provided herein are described by way of the following provided numbered arrangements and also provided as possible combinations or overlapping embodiments:

1. A retinal tamponade agent, the liquid tamponade agent comprising a structurally asymmetric partially fluorinated ether having Structure 1:

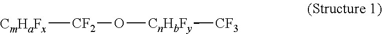

wherein m is 0, 1, or 2, or 3; a is 0, 1, or 2; x is 0, 1, 2, 3, 4, 5, or 6; n is 1 or 2, b is 1 or 2; and y is 0, 1, 2, 3, or 4.

2. The retinal tamponade agent of arrangement 1, wherein the structurally asymmetric partially fluorinated ether is chiral.

3. The retinal tamponade agent of any one of the preceding arrangements, wherein the structurally asymmetric partially fluorinated ether is asymmetric around the rotational axis.

4. The retinal tamponade agent of any one of the preceding arrangements, wherein the structurally asymmetric partially fluorinated ether changes from liquid to gaseous form at native vapor pressure.

5. The retinal tamponade agent of any one of the preceding arrangements, wherein the structurally asymmetric partially fluorinated ether has a boiling point of between about 36° C. and 58° C., and a specific gravity of less than about 1.6 g/cm³.

6. The retinal tamponade agent of any one of the preceding arrangements, wherein the structurally asymmetric partially fluorinated ether has an amplitude attenuation coefficient (db/cm-Hz) that is greater than the amplitude attenuation coefficient (db/cm-Hz) of water.

7. The retinal tamponade agent of any one of the preceding arrangements, wherein the agent comprises an amplitude attenuation coefficient (db/cm-Hz) that is greater than 0.002 db/cm-MHz.

8. The retinal tamponade agent of any one of the preceding arrangements, wherein the structurally asymmetric partially fluorinated ether comprises an amplitude attenuation coefficient (db/cm-Hz) that is between about 0.003-4 db/cm-MHz.

9 The retinal tamponade agent of any one of the preceding arrangements, wherein the structurally asymmetric partially fluorinated ether is immiscible in water.

10. The retinal tamponade agent of any one of the preceding arrangements, wherein the structurally asymmetric partially fluorinated ether is non-combustible between about 36° C. and 58° C.

11. The retinal tamponade agent of any one of the preceding arrangements, wherein the structurally asymmetric partially fluorinated ether is optically clear in liquid form.

12. The retinal tamponade agent of any one of the preceding arrangements, wherein the structurally asymmetric partially fluorinated ether is optically clear in gaseous form.

13. The retinal tamponade agent of any one of the preceding arrangements, wherein the structurally asymmetric partially fluorinated ether changes from liquid to gaseous form at native vapor pressure.

14. The retinal tamponade agent of any one of the preceding arrangements, wherein the structurally asymmetric partially fluorinated ether changes from liquid to gaseous form in about 1-3 days post administration into the vitreous chamber under biological conditions.

15. The retinal tamponade agent of any one of the preceding arrangements, wherein the structurally asymmetric partially fluorinated ether changes from liquid to gaseous form in about 1 week post administration into the vitreous chamber under biological conditions.

16. The retinal tamponade agent of any one of the preceding arrangements, wherein the structurally asymmetric partially fluorinated ether does not change from a liquid to a gaseous form unless external energy is applied.

17. The retinal tamponade agent of any one of the preceding arrangements, wherein the structurally asymmetric partially fluorinated ether is carried out of the body over within about 8 days of the change from liquid to gaseous form.

18. The retinal tamponade agent of any one of the preceding arrangements, wherein the structurally asymmetric partially fluorinated ether is carried out of the body over within about 1 month of the change from liquid to gaseous form.

19. The retinal tamponade agent of any one of the preceding arrangements, wherein the structurally asymmetric partially fluorinated ether is carried out of the body over within about 2 months of the change from liquid to gaseous form.

20. The retinal tamponade agent of any one of the preceding arrangements, wherein the structurally asymmetric partially fluorinated ether is non-toxic to intraocular structures when loft in place for greater than 1 month.

21. The retinal tamponade agent of any one of the preceding arrangements, wherein the structurally asymmetric partially fluorinated ether is non-toxic to intraocular structures when left in place for greater than 6 months.

22. The retinal tamponade agent of any one of the preceding arrangements, wherein m is 1, a is 1, x is 2, n is 1, b is 2, and y is 0.

23. The retinal tamponade agent of any one of the preceding arrangements, wherein the structurally asymmetric partially fluorinated ether comprises 2,2,2-Trifluoroethyl 1,1,2,2-tetrafluoroethyl ether.

24. The retinal tamponade agent of any one of the preceding arrangements, wherein m is 0, a is 1, x is 0, n is 2, b is 1, and y is 3.

25. The retinal tamponade agent of any one of the preceding arrangements, wherein the structurally asymmetric partially fluorinated ether comprises 1,1,2,3,3,3-Hexafluoropropyl difluoromethyl ether.

26. The retinal tamponade agent of any one of the preceding arrangements, wherein m is 2, a is 0, x is 5, n is 1, b is 1, and y is 1.

27. The retinal tamponade agent of any one of the preceding arrangements, wherein the structurally asymmetric partially fluorinated ether comprises Heptafluoropropyl 1,2,2,2-tetrafluoroethyl ether.

28. The retinal tamponade agent of any one of the preceding arrangements, wherein m is 0, a is 1, x is 0, n is 2, b is 2, and y is 2.

29. The retinal tamponade agent of any one of the preceding arrangements, wherein the structurally asymmetric partially fluorinated ether comprises Difluoromethyl 2,2,3,3,3-pentafluoropropyl ether.

30. A method of treating retinal detachment, the method comprising administering into a vitreous chamber of an eye an effective amount of the liquid tamponade agent of any one of the preceding arrangements.

31. The method of arrangement 30, wherein the retinal detachment is due to an inferior retinal tear.

32. The method of arrangement 30, wherein the retinal detachment is due to a superior retinal tear.

### EXAMPLES

### Example 1

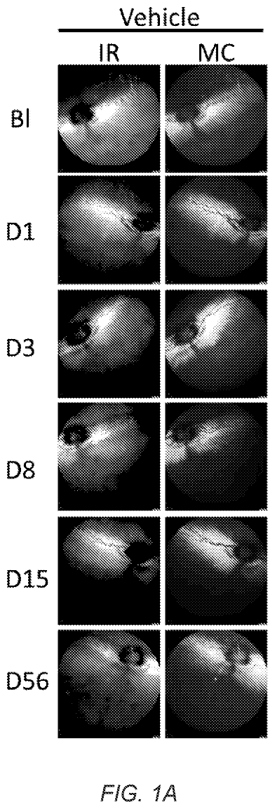

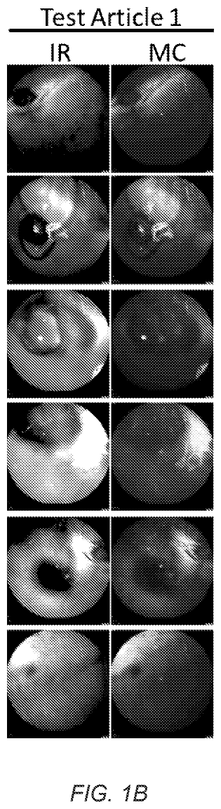

The biological safety of a compound of Structure 1 is tested within an animal model. In particular, 2,2,2-Trifluoroethyl 1,1,2,2-tetrafluoroethyl ether was tested within a New Zealand white rabbits, an animal model in line with previous retinal tamponade studies (Santos et al., Retina, 33 (1): p 120-127 (1991)). Results are shown in FIG. 1 through FIG. 7.

FIG. 1A includes a set of control retinal fundus images (near-infrared images IR and corresponding color images MC) for which balanced salt solution BSS (effectively water) was injected into a rabbit vitreous cavity. FIG. 1B includes a representative set of fundus images from a single rabbit in which 8 microliters of 2,2,2-Trifluoroethyl 1,1,2,2-tetrafluoroethyl ether (Test Article 1) were injected in a similar fashion. The horizontal rows show the progression of the bubble through time—as one moves downward on the two columns on the right, one can see the pre-injection image without a bubble (B1), the day after injection image with a clear liquid bubble posteriorly and overlying air bubble (D1), a coalesced liquid bubble posteriorly 3 days post injection (D3), a fully phase shifted gas bubble 8 days post injection (D8), a smaller gas bubble 15 days post injection (D15), and an almost fully disappeared gas bubble 56 days post injection (D56). In other words, 2,2,2-Trifluoroethyl 1,1,2,2-tetrafluoroethyl ether was empirically determined to successfully phase shift under biological conditions at around 1 week post placement, and then subsequently carried out of the body over the next 2 months. This finding is a novel one, as no such retinal tamponade with the characteristics described herein has ever been empirically shown to phase shift under biological conditions in such a manner.

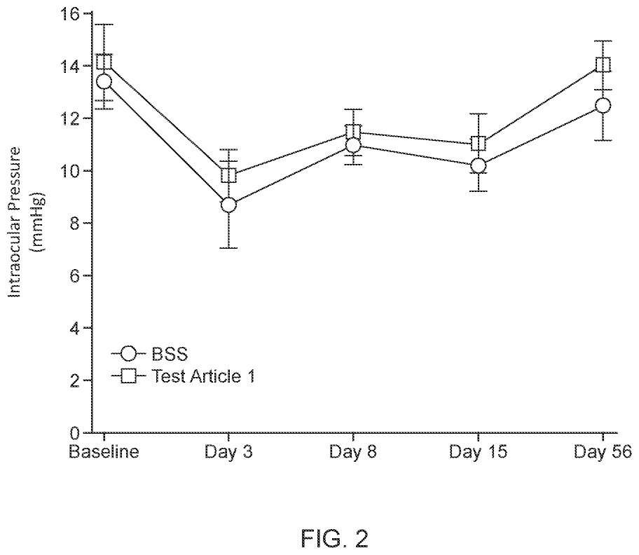

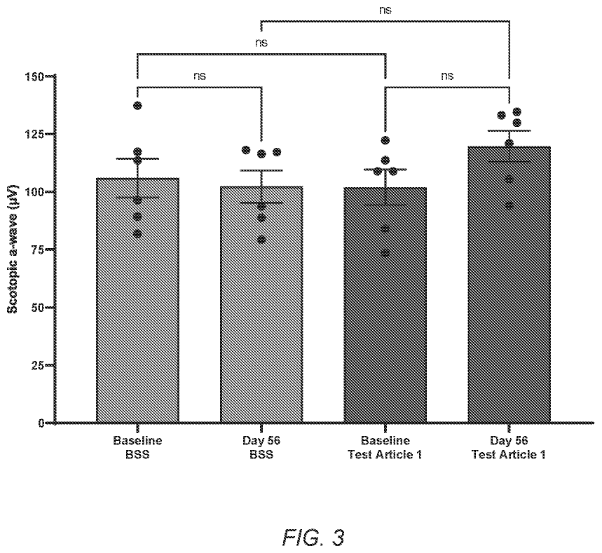

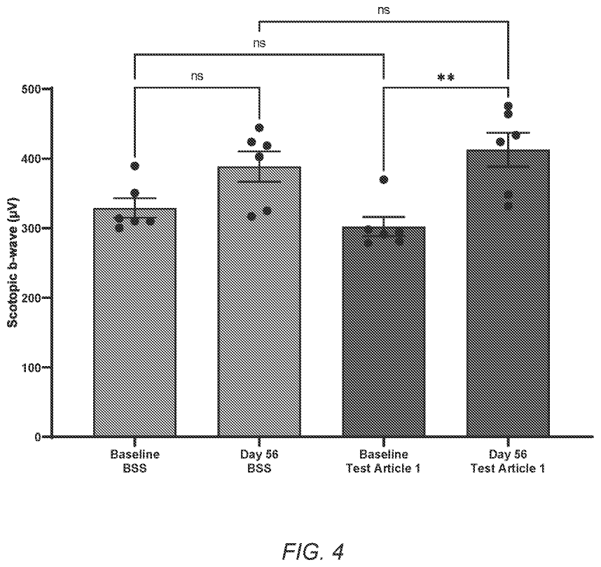

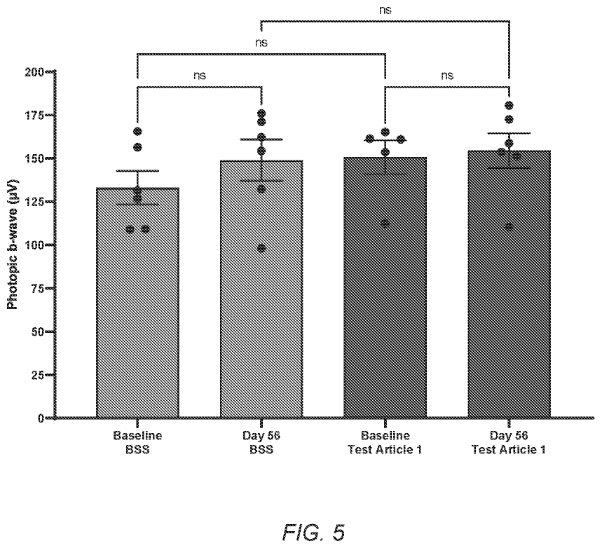

Additionally, to address concerns about biological toxicity of these compounds, intraocular pressure measurements and both scotopic a-wave, b-wave, and photopic b-wave ERGs were done on all 6 rabbits in the study, without any statistically notable adverse signals (FIG. 2-5). FIG. 2 demonstrates that the intraocular pressure between eyes injected with 2,2,2-Trifluoroethyl 1,1,2,2-tetrafluoroethyl ether (Test Article 1) was not significantly different as compared to the control (Balanced Salt Solution), nor were their intraocular pressures outside of normal physiologic range throughout the study. FIG. 3 includes a graph demonstrating no significant difference between 2,2,2-Trifluoroethyl 1,1,2,2-tetrafluoroethyl ether (Test Article 1) and the control (Balanced Salt Solution) as it pertains to electrical amplitude generation within the rod and cone cells of the retina under dark-adapted conditions at both baseline and 2 months. FIG. 4 includes a graph demonstrating no significant difference between 2,2,2-Trifluoroethyl 1,1,2,2-tetrafluoroethyl ether (Test Article 1) and the control (Balanced Salt Solution) as it pertains to electrical amplitude generation within the bipolar cells of the retina under dark-adapted conditions at both baseline and 2 months. FIG. 5 includes a graph demonstrating no significant difference between 2,2,2-Trifluoroethyl 1,1,2,2-tetrafluoroethyl ether (Test Article 1) and the control (Balanced Salt Solution) as it pertains to electrical amplitude generation within the bipolar cells of the retina under light-adapted conditions at both baseline and 2 months.

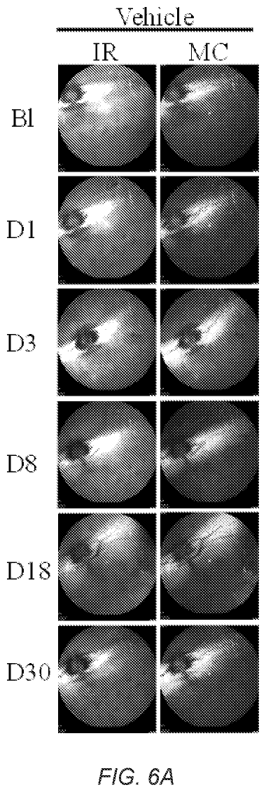

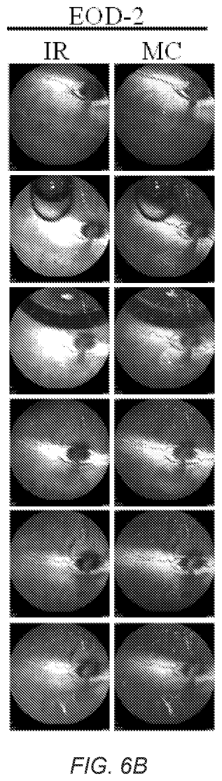

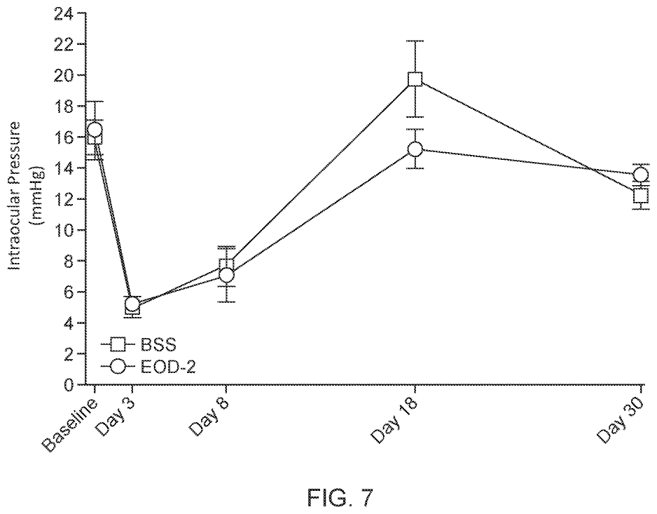

Similarly, the biological safety of another compound of Structure 1 is tested within an animal model. In particular, 1,1,2,3,3,3-Hexafluoropropyl difluoromethyl ether was tested within a New Zealand white rabbits, an animal model in line with previous retinal tamponade studies (Santos et al., Retina, 33 (1): p 120-127 (1991)). Results are shown in FIG. 6 and FIG. 7.

FIG. 6A includes a set of control retinal fundus images (near-infrared images IR and corresponding color images MC) for which balanced salt solution BSS (effectively water) was injected into a rabbit vitreous cavity. FIG. 6B includes a representative set of fundus images from a single rabbit in which 8 microliters of 1,1,2,3,3,3-Hexafluoropropyl difluoromethyl ether (EOD-2) were injected in a similar fashion. The horizontal rows show the progression of the bubble through time—as one moves downward on the two columns on the right, one can see the pre-injection image without a bubble (B1), the day after injection image with a clear liquid bubble posteriorly and overlying air bubble (D1), a fully phase shifted gas bubble posteriorly 3 days post injection (D3), a fully disappeared bubble 8 days post injection (D8), which does not return at either of the subsequent timepoints (D18 and D30). In other words, 1,1,2,3,3,3-Hexafluoropropyl difluoromethyl ether was empirically determined to successfully phase shift under biological conditions at around 1-3 days post placement, and then subsequently fully carried out of the body by Day 8. While similar to the previous example, this chemical evaporates and is removed from the eye much more quickly.

Additionally, to address concerns about biological toxicity of these compounds, intraocular pressure measurements were done on all 4 rabbits in the study, without any statistically notable adverse signals. FIG. 7 demonstrates that the intraocular pressure between eyes injected with 1,1,2,3,3,3-Hexafluoropropyl difluoromethyl ether (EOD-2) was not significantly different as compared to the control (Balanced Salt Solution), nor were their intraocular pressures outside of normal physiologic range throughout the study.

Finally, in addition to all of the previously noted criteria for intraocular use (combustible stability, phase-shifting capability at biological conditions, optical clarity, increased ultrasonic absorption, immiscibility, and biological nontoxicity) useful chemicals must also possess a safe density. Previous accounts have noted that prolonged exposure of the retina to a liquid tamponade with an elevated specific gravity (greater than or equal to 1.6 g/ml) can cause retinal thinning and atrophy (Chang et al., Retina, 11:367-374 (1991)). Hence, those with specific gravities that are lower than the threshold for damage seen can be considered medically useable in this context (less than or equal to about 1.6 g/ml). Table 5 below demonstrates various fluorinated ethers and their specific gravities:

TABLE 5
Densities of Selected Fluorinated Ethers

| | Chemical | Density | Boiling |
|---|---|---|---|
| Chemical Name | Formula | (g/cm³) | Point (° C.) |
| 1,1,2,3,3,3-Hexafluoropropyl | C₄H₂F₈O | 1.5 | 47 |
| difluoromethyl ether | | | |
| Difluoromethyl 2,2,3,3,3- | C₄H₃F₇O | 1.46 | 46 |
| pentafluoropropyl ether | | | |
| 2,2,2-Trifluoroethyl 1,1,2,2- | C₄H₃F₇O | 1.487 | 56.7 |
| tetrafluoroethyl ether | | | |
| heptafluoropropyl 1,2,2,2- | C₅HF₁₁O | 1.538 | 40 |
| tetrafluoroethyl ether | | | |
| 1,1,2,2-Tetrafluoro-3-(2,2,3,3- | C₆H₆F₈O | 1.627 | 35 |
| tetrafluoropropoxy )propane | | | |
| 1,1,1,2,2,3,3-Heptafluoro- | C₇H₃F₁₃O | 1.63 | 102 |
| 4-(1,1,2,3,3,3- | | | |
| hexafluoropropoxy)butane | | | |

The above table demonstrates that it is non-trivial to find a fluorinated ether that matches the criterion noted herein. Indeed, even some partially fluorinated ethers noted previously, such as 1,1,1,2,2,3,3-Heptafluoro-4-(1,1,2,3,3,3-hexafluoropropoxy) butane, often do not have an in-range boiling point or do not conform to having an in-range density.

1. A retinal tamponade agent comprising a structurally asymmetric partially fluorinated ether having Structure 1: 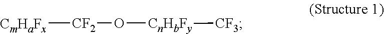
wherein m is 0, 1, or 2, or 3; a is 0, 1, or 2; x is 0, 1, 2, 3, 4, 5, or 6; n is 1 or 2, b is 1 or 2; and y is 0, 1, 2, 3, or 4.

2. The retinal tamponade agent of claim 1, wherein the structurally asymmetric partially fluorinated ether is chiral.

3. The retinal tamponade agent of claim 1, wherein the structurally asymmetric partially fluorinated ether is asymmetric around a rotational axis.

4. The retinal tamponade agent of claim 1, wherein the structurally asymmetric partially fluorinated ether changes from liquid to gaseous form at native vapor pressure.

5. The retinal tamponade agent of claim 1, wherein the structurally asymmetric partially fluorinated ether has a boiling point of between about 36° C. and 58° C., and a specific gravity of less than about 1.6 g/cm³.

6. (canceled)

7. The retinal tamponade agent of claim 1, wherein the agent comprises an amplitude attenuation coefficient (db/cm-Hz) that is greater than 0.002 db/cm-MHz.

8. The retinal tamponade agent of claim 7, wherein the structurally asymmetric partially fluorinated ether comprises an amplitude attenuation coefficient between about 0.003 and about 4 db/cm-MHz.

9. The retinal tamponade agent of claim 1, wherein the structurally asymmetric partially fluorinated ether is immiscible in water.

10. The retinal tamponade agent of claim 1, wherein the structurally asymmetric partially fluorinated ether is non-combustible between about 36° C. and 58° C.

11. The retinal tamponade agent of claim 1, wherein the structurally asymmetric partially fluorinated ether is optically clear in liquid and/or gaseous form.

12. (canceled)

13. (canceled)

14. The retinal tamponade agent of claim 1, wherein the structurally asymmetric partially fluorinated ether changes from liquid to gaseous form in up to about 1 week post administration into the vitreous chamber under biological conditions.

15. The retinal tamponade agent of claim 1, wherein the structurally asymmetric partially fluorinated ether changes from a liquid to a gaseous form when an external energy is applied.

16. (canceled)

17. (canceled)

18. The retinal tamponade agent of claim 1, wherein the structurally asymmetric partially fluorinated ether is carried out of the body within about 2 months of the change from liquid to gaseous form.

19. The retinal tamponade agent of claim 1, wherein the structurally asymmetric partially fluorinated ether is non-toxic to intraocular structures when left in place for greater than 1 month.

20. (canceled)

21. The retinal tamponade agent of claim 1, wherein m is 1, a is 1, x is 2, n is 1, b is 2, and y is 0.

22. The retinal tamponade agent of claim 1, wherein the structurally asymmetric partially fluorinated ether comprises 2,2,2-Trifluoroethyl 1,1,2,2-tetrafluoroethyl ether, 1,1,2,3,3,3-Hexafluoropropyl difluoromethyl ether, Heptafluoropropyl 1,2,2,2-tetrafluoroethyl ether, or Difluoromethyl 2,2,3,3,3-pentafluoropropyl ether.

23. The retinal tamponade agent of claim 1, wherein m is 0, a is 1, x is 0, n is 2, b is 1, and y is 3.

24. (canceled)

25. The retinal tamponade agent of claim 1, wherein m is 2, a is 0, x is 5, n is 1, b is 1, and y is 1.

26. (canceled)

27. The retinal tamponade agent of claim 1, wherein m is 0, a is 1, x is 0, n is 2, b is 2, and y is 2.

28. (canceled)

29. A method of treating retinal detachment, the method comprising administering into a vitreous chamber of an eye an effective amount of the retinal tamponade agent of claim 1.

30. (canceled)

31. (canceled)
